(19) United States
(12) Patent Application Publication
(10) Pub. No.: US 2004/0168264 A1
(43) Pub. Date: Sep. 2, 2004

(54) Hair colourants

(76) Inventors: Hendrick Petrus Frick, Ayrshire (ZA); Ajit Kumar, West Midlands (GB)

Correspondence Address: Huntsman, Legal Department, P O Box 15730, Austin, TX, 78761, US

(21) Appl. No.: 10/480,228

(22) PCT Filed: Jun. 10, 2002
(86) PCT No.: PCT/EP02/06302

(30) Foreign Application Priority Data
Jun. 13, 2001 (GB) 0114372.6

(51) Int. Cl. A61K 7/13

(52) U.S. Cl. 8/405

(57) ABSTRACT

A composition comprising a cationic dye which is substantive to hair and amphoteric surfactant wherein said amphoteric surfactant consists, at least predominantly of a betaine, preferably a C₁₂₋₂₅ alkyl amidopropyl betaine, and the formulation contains less than 15% by weight of sodium chloride based on the weight of betaine.

This invention relates to hair colourant formulations. Such formulations typically comprise a cationic dye, which is substantive to hair, and a surfactant. The surfactant cannot be anionic due to the incompatibility of anionic surfactants with cationic dyes. Cationic surfactants are substantive to hair and compete with the dye. Therefore the only surfactants that can be used are non-ionic and amphoteric, which latter term is used herein to include zwitterionic surfactants.

The common amphoteric surfactants are betaines, which are made by reacting sodium chloracetate with an amine. The reaction forms sodium chloride as an equimolar by-product. Betaines therefore contain about 20% by weight of salt based on the weight of surfactant. However it has been found that salt inhibits the uptake of dye by hair. For this reason the use of betaines has been avoided in hair dyeing applications and most hair dyeing formulations are based on non-ionic surfactants. An exception to this is the amino propionates which are amphoteric surfactants made by reacting amines with acrylic acid, and which therefore contain no salt. They have been successfully used in hair colourant formulations:

We have now found that certain betaines, from which the salt has been removed, are substantially more effective than either amino propionates or non-ionic surfactants in promoting the uptake of dye by hair.

The invention provides a composition comprising a cationic dye which is substantive to hair and amphoteric surfactant wherein said amphoteric surfactant consists, at least predominantly of a betaine, preferably a C₁₂₋₂₅ alkyl amidopropyl betaine, and the formulation contains less than 15% by weight of sodium chloride based on the weight of betaine.

We strongly prefer that the amount of salt should be less than 10% more preferably less than 5%, especially less than 3%, most preferably less than 1% e.g. less than 0.5% based on the weight of betaine.

Sodium ion may be removed from amphoteric surfactants, either by electrosmosis, e.g. as described in our GB 1 525 692 or in EP 0 736 521, or by membrane filtration, for example as described in EP 0 626 881, or, less preferably, by displacing sodium ion with, for example potassium or ammonium, e.g. using ion exchange.

The preferred betaine is coconut amido propyl betaine commonly know as CAPB. The term CAPB is normally used generically to include amido propyl betaines made from coconut oil feedstocks and also from other feedstocks, natural or synthetic with a similar distribution of homologues, e.g. palm oil. CAPB comprises C₁₂ amido propyl betaine as its principal constituent with smaller amounts of the C₈, C₁₀, C₁₄, C₁₆, and C₁₈ alkyl and oleyl homologues. Hair colourant compositions are supplied as aqueous solutions since anhydrous non-ionic surfactants are irritant liquids and amphoteric surfactants are strongly hygroscopic.

We have discovered, however, that certain betaines, and especially low salt amido propyl betaines, such as lauryl amido propyl betaine (LAPB), which is a refined form of CAPB containing more than 80% of the C₁₂ homologue, can be dried to form a non-hygroscopic powder or granular solid. Surprisingly cationic hair dyes have been found to adsorb strongly onto the particles of betaine.

According to a preferred aspect therefore our invention provides a solid hair colourant comprising particles of a non-hygroscopic betaine, preferably an amido propyl betaine, containing less than 15% by weight of sodium chloride and less than 12% by weight of water based on the weight of betaine, and a cationic dye, which is substantive to hair, adsorbed on said particles.

The betaines used in conjunction with the aforesaid preferred aspect are preferably alkyl amidopropyl betaines, wherein said alkyl consists of at least 85%, preferably at least 90%, by weight thereof, of a single homologue with from 12 to 25 carbon atoms. Preferably the betaine contains less than 10%, e.g. 2 to 8%, by weight of moisture.

The solid product may conveniently be prepared by dry mixing the solid surfactant and dye powder, e.g. by mixing finely powdered dye with a granular betaine of larger particle size, agitating the mixture and sieving to remove any excess dye. It may be compressed into tablets, which are readily soluble in water to provide a dye solution when required.

The proportion of dye is typically between 0.05 and 10% based on the total weight of the composition preferably 0.1 to 5% more preferably 0.5 to 3%. The proportion of betaine may typically be from 5 to 30% by weight in aqueous formulations, preferably 10 to 20%, or 90 to 99.9% of solid formulations.

The aqueous formulations may optionally contain minor proportions of non-ionic surfactant, especially amine oxide, or alkyl polyglucoside and of polymeric thickener. The latter may be used in whatever proportion is required to give the desired consistency from liquid dye to mousse. The pH of the composition at its working dilution should be from 6 to 8.5 preferably 7 to 7.5.

The invention is illustrated by the following examples, in which references to “desalted” relate to betaines containing less than 1%, by weight, based on the weight of amphoteric surfactant, of sodium chloride, and all % are by weight based on the composition unless stated otherwise.

### EXAMPLE 1

A liquid hair dye comprised

| | |
|---|---|
| 1.75% | coconut amido propyl amine oxide. |
| 12.88% | CAPB (desalted) |
| 1% | polymeric thickener |
| 1% | basic brown 17 |
| Balance | water |

The pH was adjusted to 7-7.5

The above formulation gave substantially improved uptake of dye on hair compared with a similar formulation containing an amino propionate.

### EXAMPLE 2

Desalted LAPB (98% C₁₂ homologue, based on total amphoteric) was vacuum dried to a moisture content of 6%, based on surfactant, and the granular product was dry mixed with basic brown 17. The granules were separated from the excess powder to provide a free flowing granular product containing 1% dye. The granules were compacted into tablets under a pressure of 1000N. The surface of the tablets was moistened with water to provide a shiny finish.

1. A composition comprising a cationic dye which is substantive to hair and amphoteric surfactant wherein said amphoteric surfactant consists, at least predominantly of a betaine and the formulation contains less than 15% by weight of sodium chloride based on the weight of betaine.

2. A composition according to claim 1, wherein said betaine is a C₁₂₋₁₆ alkyl amidopropyl betaine.

3. A composition according to either of claims 1 and 2 containing less than 5%, by weight, based on the weight of amphoteric surfactant, of sodium chloride.

4. A solid hair colourant comprising particles of a non-hygroscopic betaine, containing less than 15% by weight of sodium chloride and less than 12% by weight of water based on the weight of betaine, and a cationic dye, which is substantive to hair, adsorbed on said particles.

5. A colourant according to 4, wherein the betaine is an amido propyl betaine.

6. A method of preparing a colourant according to either of claims 4 and 5, which comprises dry mixing said dye with non-hygroscopic particles of solid betaine.
